(19) United States
(12) Patent Application Publication
(10) Pub. No.: US 2009/0093652 A1
(43) Pub. Date: Apr. 9, 2009

(54) Crystalline forms cinacalcet fumarate and cinacalcet succinate and processes for preparation thereof

(76) Inventors: Michal Rafilovich, Petach-Tikva (IL); Amihai Eisenstadt, Petach-Tikva (IL)

Correspondence Address: KENYON & KENYON LLP, ONE BROADWAY, NEW YORK, NY, 10004, US

(21) Appl. No.: 12/229,045

(22) Filed: Aug. 18, 2008

Related U.S. Application Data
(60) Provisional application No. 60/965,111, filed on Aug. 16, 2007.

(51) Int. Cl. C07C 69/34; C07C 69/52; A61K 31/225

(52) U.S. Cl. 560/194; 514/548

(57) ABSTRACT

The present invention provides crystalline forms of Cinacalcet Fumarate and Cinacalcet Succinate, pharmaceutical compositions comprising the crystalline form of Cinacalcet Fumarate and/or the crystalline form of Cinacalcet Succinate, and processes for preparing the crystalline forms of Cinacalcet Fumarate and Cinacalcet Succinate and pharmaceutical compositions comprising the crystalline forms.

### CROSS REFERENCE TO RELATED APPLICATIONS

The present application claims the benefit of the following U.S. Provisional Patent Application No. 60/965,111, filed Aug. 16, 2007. The content of this application is incorporated herein by reference.

### FIELD OF THE INVENTION

The present invention relates to solid state chemistry of Cinacalcet. In particular, the present invention is directed to crystalline forms of Cinacalcet Fumarate and Cinacalcet Succinate and to methods of preparing the crystalline forms.

### BACKGROUND OF THE INVENTION

(R)-α-methyl-N-[3-[3-(trifluoromethyl)phenyl]propyl]-1-naphthalenemethane amine (herein “Cinacalcet” or “CNC”) has a CAS number of 226256-56-0, a formula of C₂₂H₂₂F₃N and the following structure:

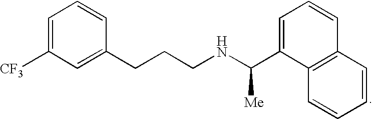

Cinacalcet is the free base form of Cinacalcet hydrochloride (herein “CNC—HCl”), which has a CAS number of 364782-34-3 and the following structure:

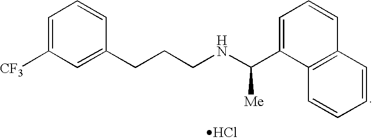

CNC—HCl is marketed as SENSIPAR™, and was the first drug in a class of compounds known as calcimimetics to be approved by the FDA. Calcimimetics are a class of orally active, small molecules that decrease the secretion of parathyroid hormone (“PTH”) by activating calcium receptors. The secretion of PTH is normally regulated by the calcium-sensing receptor. Calcimimetics increase the sensitivity of this receptor to calcium, which inhibits the release of parathyroid hormone, and lowers parathyroid hormone levels within a few hours. Calcimimetics are used to treat hyperparathyroidism, a condition characterized by the over-secretion of PTH that results when calcium receptors on parathyroid glands fail to respond properly to calcium in the bloodstream. Elevated levels of PTH, an indicator of secondary hyperparathyroidism, are associated with altered metabolism of calcium and phosphorus, which can result in bone pain, fractures, and an increased risk for cardiovascular death. As a calcimimetic, CNC—HCl is approved for the treatment of secondary hyperparathyroidism in patients with chronic kidney disease, who are on dialysis. Treatment with CNC—HCl lowers serum levels of PTH as well as the calcium/phosphorus ion product, a measure of the amount of calcium and phosphorus in the blood.

The discovery of new solid states of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional processes for preparation of pharmaceutically acceptable salts of Cinacalcet either in a crystalline or in an amorphous form.

### SUMMARY

The present invention relates to Cinacalcet Fumarate and Cinacalcet Succinate. Preferably, the Cinacalcet Fumarate and Cinacalcet Succinate are solid, more preferably crystalline.

The present invention provides a crystalline form of Cinacalcet Fumarate characterized by a data selected from a powder XRD pattern with two peaks at about 7.5 and 19.4±0.2 degrees two-theta and three peaks selected from a list of five peaks at about 11.6, 12.6, 16.3, 17.2 and 25.4±0.2 degrees two-theta; and a powder XRD pattern with main peaks at about 7.5, 15.1, 16.3, 18.1° 2θ±0.2° 2θ.

The present invention also provides a process for preparing the crystalline form of Cinacalcet Fumarate comprising combining Cinacalcet with fumaric acid in the presence of organic solvent.

The present invention also provides a crystalline form of Cinacalcet Succinate characterized by a data selected from a powder XRD pattern with two peaks at about 6.7 and 18.9±0.2 degrees two-theta and three peaks selected from a list of five peaks at about 7.2, 10.7, 14.4, 16.3 and 17.0±0.2 degrees two-theta; and a powder XRD pattern with main peaks at about 6.7, 7.2, 13.5, 15.1° 2θ±0.2° 2θ.

The present invention also provides a process for preparing the crystalline form of Cinacalcet Succinate comprising combining succinic acid with cinacalcet in the presence of organic solvent to obtain a mixture; heating the mixture; and recovering the precipitate.

The present invention further comprises a pharmaceutical composition comprising the crystalline form of Cinacalcet Fumarate and/or Cinacalcet Succinate of the present invention and at least one pharmaceutically acceptable excipient.

The present invention comprises a pharmaceutical composition comprising the crystalline form of Cinacalcet Fumarate and/or Cinacalcet Succinate made by the processes of the present invention, and at least one pharmaceutically acceptable excipient.

The present invention further encompasses a process for preparing a pharmaceutical formulation comprising combining the crystalline form of Cinacalcet Fumarate and/or Cinacalcet Succinate of the present invention with at least one pharmaceutically acceptable excipient.

The present invention further encompasses the use the crystalline form of Cinacalcet Fumarate and/or Cinacalcet Succinate for the manufacture of a pharmaceutical composition.

### BRIEF DESCRIPTION OF THE FIGURES

FIG. 1 shows a powder X-ray diffraction pattern for Cinacalcet Fumarate Form A.

FIG. 2 shows a powder X-ray diffraction pattern for Cinacalcet Succinate Form A.

FIGS. 3a, and 3b show the ¹H NMR spectrum of Cinacalcet Fumarate.

FIGS. 4a, 4b, and 4c show the ¹³C NMR spectrum of Cinacalcet Fumarate.

FIGS. 5a, and 5b show the ¹H NMR spectrum of Cinacalcet Succinate.

FIGS. 6a, 6b, and 6c show the ¹³C NMR spectrum of Cinacalcet Succinate.

### DETAILED DESCRIPTION

As used herein, the term “room temperature” refers to a temperature of about 20° C. to about 30° C.

The present invention relates to Cinacalcet Fumarate and Cinacalcet Succinate. Preferably, the Cinacalcet Fumarate and Cinacalcet Succinate are solid, more preferably crystalline.

The present invention provides a crystalline form of Cinacalcet Fumarate characterized by a data selected from a powder XRD pattern with two peaks at about 7.5 and 19.4±0.2 degrees two-theta and three peaks selected from a list of five peaks at about 11.6, 12.6, 16.3, 17.2 and 25.4±0.2 degrees two-theta; and a powder XRD pattern with main peaks at about 7.5, 15.1, 16.3, 18.1° 2θ±0.2° 2θ.

The crystalline form of Cinacalcet Fumarate may be further characterized by a powder XRD pattern with an additional peak at about 24.5±0.2 degrees two-theta.

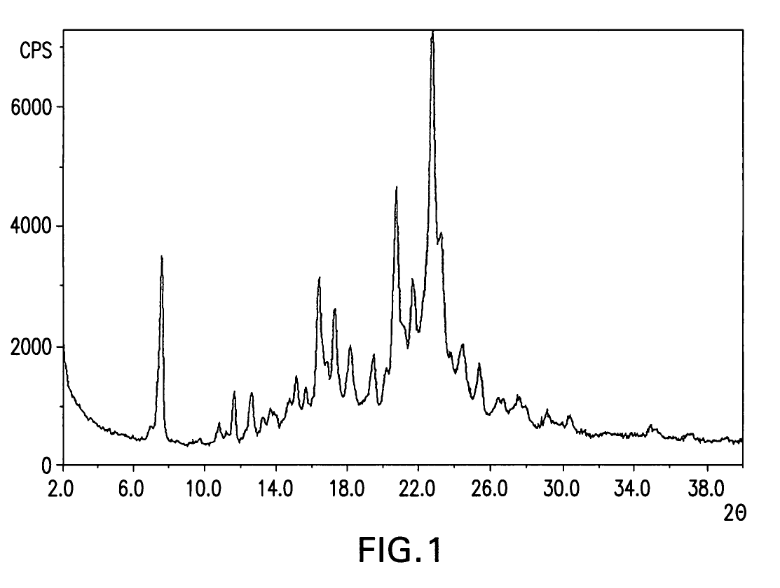

The crystalline form of Cinacalcet Fumarate may be further characterized by a powder XRD pattern substantially as illustrated in FIG. 1.

The present invention also provides a process for preparing the crystalline form of Cinacalcet Fumarate by reacting cinacalcet with fumaric acid in an organic solvent. Typically, precipitation begins almost instantaneously after Cinacalcet and fumaric acid are combined.

Examples of organic solvents include C₃ to C₇ esters, C₁ to C₆ chlorinated hydrocarbons, C₆ to C₁₂ aromatic hydrocarbons. Specific examples of such solvents include ethyl acetate, dichloromethane, toluene, and chloroform. The most preferred solvent is ethyl acetate.

In a specific embodiment, the process first comprises neutralizing a Cinacalcet salt with a base. The base is preferably an inorganic base, such as an alkali metal hydrogen carbonate, such as sodium bicarbonate, an alkali metal carbonate, or an alkali hydroxide. The most preferred base is sodium bicarbonate. The Cinacalcet salt can be Cinacalcet HCl, Cinacalcet HBr, Cinacalcet sulfate, or Cinacalcet phosphate. Preferably the salt is Cinacalcet HCl.

Preferably after combining of the salt and the base, the reaction mixture is then stirred for about 15 minutes to about 1.5 hours, preferably for about an hour. Preferably, the reaction mixture is stirred at a temperature of from about 15° C. to about 55° C., more preferably, at room temperature.

The molar ratio of fumaric acid to cinacalcet is preferably about 1:1 to about 1:1.2. The ratio of organic solvent to cinacalcet base is preferably about 5:1 to about 7:1 (v/w)

The resulting Cinacalcet Fumarate can then be recovered using any method known in the art, such as filtration and optionally washed with an organic solvent, such as those listed above, which may be the same or different from the solvent used in the reaction of Cinacalcet with fumaric acid.

The above process can be carried out in one pot. In this process, a salt of cinacalcet as described above, preferably cinacalcet HCl, is neutralized with a base, in the presence of an organic solvent, and then reacted with fumaric acid in the same solvent. The organic solvent used in this process is as described above.

The present invention also provides a crystalline form of Cinacalcet Succinate characterized by data selected from a powder XRD pattern with two peaks at about 6.7 and 18.9±0.2 degrees two-theta and three peaks selected from a list of five peaks at about 7.2, 10.7, 14.4, 16.3 and 17.0±0.2 degrees two-theta; and a powder XRD pattern with main peaks at about 6.7, 7.2, 13.5, 15.1° 2θ±0.2° 2θ.

The crystalline form of Cinacalcet Succinate may be further characterized by a powder XRD pattern with an additional peak at about 18.5° 2θ±0.2° 2θ.

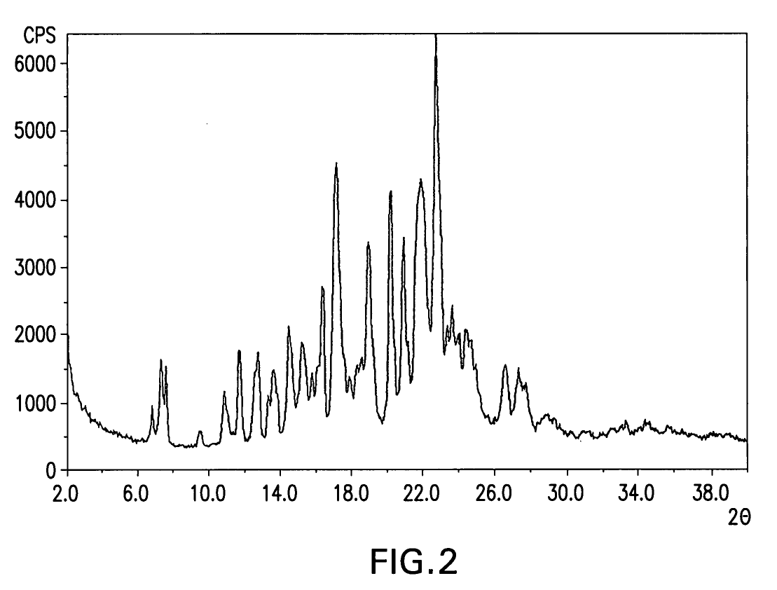

The crystalline form of Cinacalcet Succinate may be further characterized by a powder XRD pattern substantially as illustrated in FIG. 2.

The present invention also encompasses a process for preparing the crystalline form of Cinacalcet Succinate comprising combining succinic acid with cinacalcet in the presence of organic solvent to obtain a mixture; heating the mixture; and recovering the precipitate.

In a specific embodiment, the process first comprises neutralizing a Cinacalcet salt with a base. The base can be either in a solid form or in aqueous solution. The base is preferably an inorganic base, such as an alkali metal hydrogen carbonate, such as sodium bicarbonate, such as aqueous NaHCO₃, an alkali metal carbonate, or an alkali hydroxide. The most preferred base is aqueous NaHCO₃. The Cinacalcet salt is preferably Cinacalcet HCl, Cinacalcet HBr Cinacalcet sulfate, or Cinacalcet phosphate. More preferably the salt is Cinacalcet HCl.

A water immiscible organic solvent is added to the reaction mixture, preferably after neutralization, so that the cinacalcet base would react with succinic acid and move into the solvent. Examples of organic solvents include C₃ to C₇ esters, C₁ to C₆ chlorinated hydrocarbons, C₆ to C₁₂ aromatic hydrocarbons. Specific examples of such solvents include ethyl acetate, dichloromethane, toluene, and chloroform.

If an aqueous solution of base is used with a water immiscible organic solvent, the reaction mixture will form aqueous and organic layers. The layers are preferably separated. The aqueous layer is then preferably extracted with an organic solvent that may be the same or different as the solvent used in the neutralization. The combined organic layers may then be heated if necessary to dissolve all solids. The combined organic layers can be heated at a temperature of from about 15° C. to about 55° C., more preferably at a temperature of about 45° C.

The molar ratio of succinic acid to cinacalcet is preferably about 1:1 to about 1:1.2. The ratio of organic solvent to cinacalcet base is preferably about 5:1 to about 7:1 (v/w).

Preferably, the reaction mixture of Cinacalcet base and the succinic acid is stirred for about 0.5 hours to about 1.5 hours, preferably for about an hour. Preferably, the reaction mixture is stirred at a temperature of from about 35° C. to about 55° C., more preferably, at 45° C.

Preferably, the mixture is further evaporated; and the salt is precipitated from ether.

The cinacalcet succinate can be recovered from the reaction mixture. Recovery can be done by concentrating the reaction mixture if the reaction mixture is too dilute. Concentration can be done by removing the solvent, preferably by evaporation. Evaporation can be carried out under a pressure of less than one atmosphere, or less than 100 mmHg. A rotary evaporator can be used. If crystallization does not occur, additional solvent can be added, and the vessels of the reaction mixture can be scratched. The resulting precipitate can be separated from the reaction mixture by filtration.

The recovered crystals can be dried. Drying can be carried out under a pressure of less than one atmosphere, or less than 100 mmHg, most preferably less than 25 mmHg at about 20° C. to about 30° C. for about 12 hours to about 24 hours.

The above process can be carried out in one pot. In this process, a salt of cinacalcet as described above, preferably cinacalcet HCl, is neutralized with a base in the presence of an organic solvent, and then reacted with succinic acid in the same solvent. The organic solvent used in this process is as described above.

The present invention further provides a pharmaceutical composition comprising the crystalline form of Cinacalcet Fumarate and/or Cinacalcet Succinate of the present invention and at least one pharmaceutically acceptable excipient.

The present invention further provides a pharmaceutical composition comprising the crystalline form of Cinacalcet Fumarate and/or Cinacalcet Succinate, made by the processes of the present invention, and at least one pharmaceutically acceptable excipient.

The present invention further encompasses a process for preparing a pharmaceutical formulation, comprising combining the crystalline form of Cinacalcet Fumarate and/or Cinacalcet Succinate of the present invention with at least one pharmaceutically acceptable excipient.

The present invention further encompasses the use of the crystalline form of Cinacalcet Fumarate and/or Cinacalcet Succinate for the manufacture of a pharmaceutical composition.

Methods of administration of a pharmaceutical composition of the present invention preferably comprise administration in various preparations, depending on the age, sex, and symptoms of the patient. The pharmaceutical compositions are preferably administered, for example, as tablets, pills, powders, liquids, suspensions, emulsions, granules, capsules, suppositories, injection preparations (solutions and suspensions), and the like. When the pharmaceutical composition is a liquid that comprises any one of the crystalline forms of the present invention, the liquid pharmaceutical composition is a suspension or emulsion, wherein the Cinacalcet Fumarate and/or Cinacalcet Succinate retains its crystalline form.

Pharmaceutical compositions of the present invention can optionally be mixed with other forms of Cinacalcet and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents may include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc, and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the crystalline Cinacalcet Fumarate and/or Cinacalcet Succinate of the present invention is suspended, retaining its crystalline form, together with and any other solid ingredients, which may be dissolved or suspended, in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain viscosity enhancing agents to improve the mouth-feel of the product and/or coat the lining of the gastrointestinal tract. Such agents include, for example, acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents, such as sorbitol, saccharin, sodium saccharin, sucrose, aspartame, fructose, mannitol, and invert sugar can be added to improve the taste.

Preservatives and chelating agents, such as alcohol, sodium benzoate, butylated hydroxy toluene, butylated hydroxyanisole, and ethylenediamine tetraacetic acid can be added at safe levels to improve storage stability.

A liquid pharmaceutical composition according to the present invention can also contain a buffer, such as guconic acid, lactic acid, citric acid or acetic acid, sodium guconate, sodium lactate, sodium citrate, or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting without being subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized, and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations during the treatment of schizophrenia.

The amount of Cinacalcet of the present invention contained in a pharmaceutical composition according to the present invention is not specifically restricted. However, the dose should be sufficient to treat, ameliorate, or reduce the condition.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Instruments

### XRD

Powder X-ray diffraction (“XRD”) analysis can be carried out using any XRD powder diffractometer commonly used in the industry. The samples of this invention were run in a SCINTAG powder X-ray diffractometer model X'TRA equipped with a solid-state detector. Copper radiation of λ=1.5418 Å. The sample can be introduced using a round standard aluminum sample holder with round zero background quartz plate in the bottom and is scanned by a continuous scan at a rate of 3° per minute.

### Preparation of a Crystalline Form of Cinacalcet Fumarate.

### Example 1

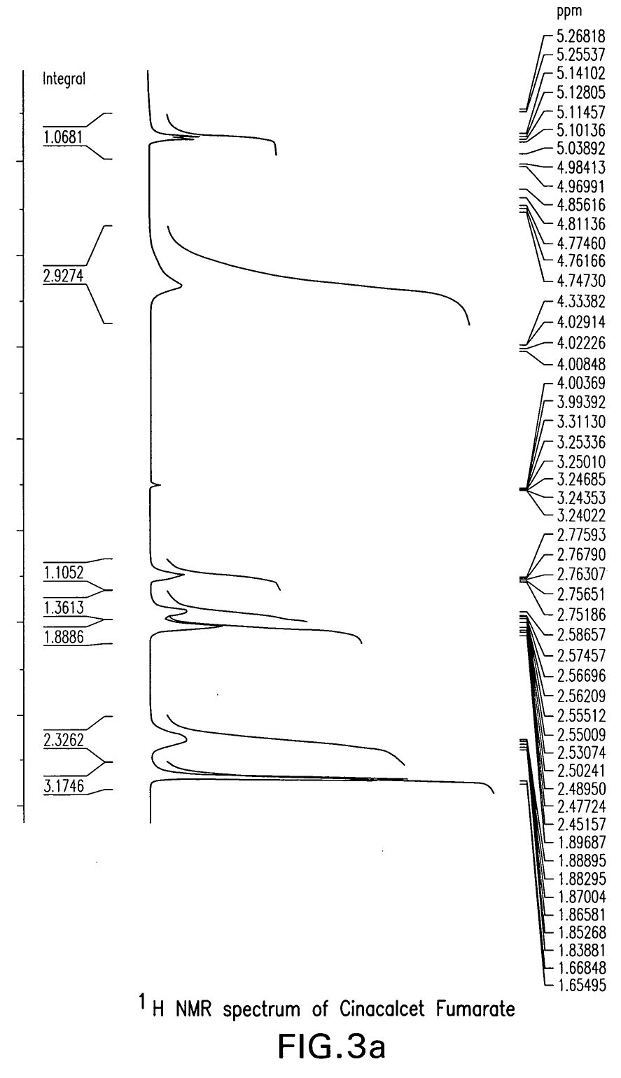

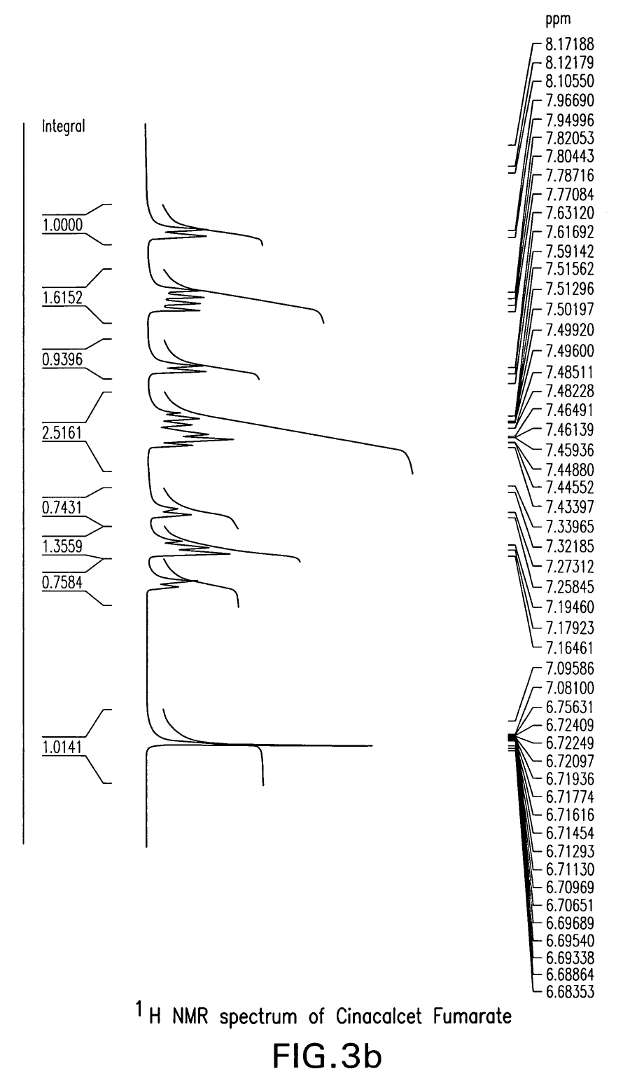

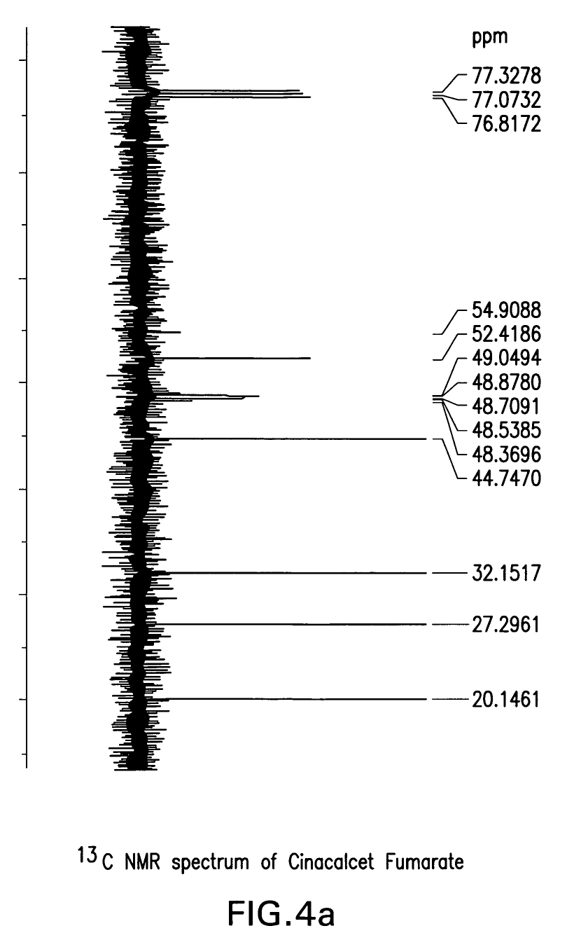

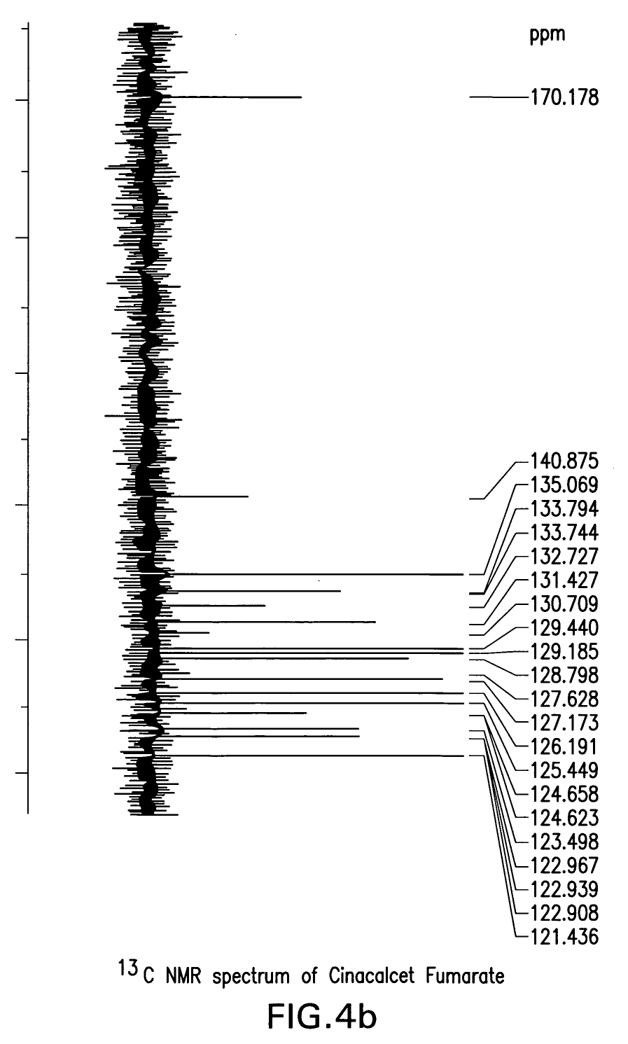

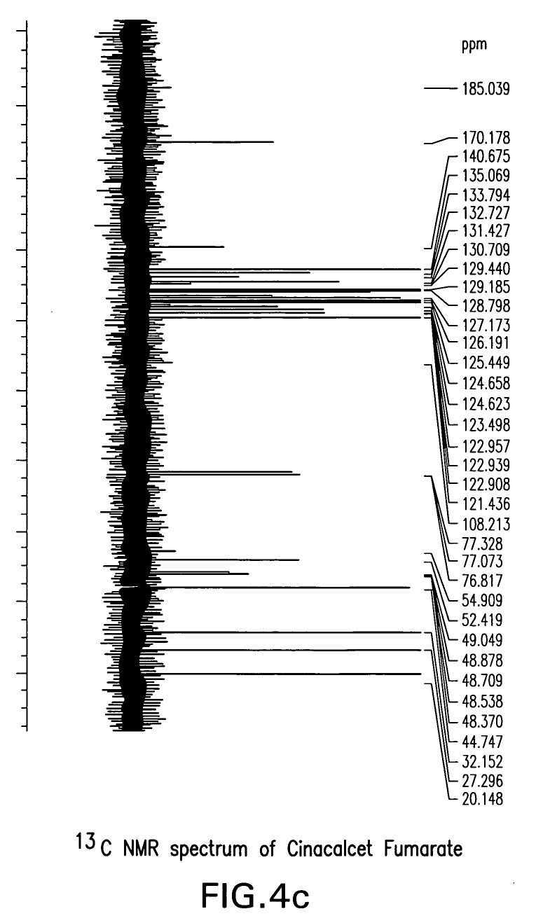

3.97 g cinacalcet hydrochloride (10 mmole) was stirred with 20 ml of NaHCO₃ (5% in water) and 10 ml of ethyl acetate at room temperature for 1 hour. The layers were separated and the aqueous solution was further extracted twice with 5 ml ethyl acetate each. The combined organic solution was passed through a Hi-flow pad, and treated with 1.14 g fumaric acid in 20 ml ethyl acetate, precipitation started almost instantaneously. The reaction mixture was stirred for 1 hour at room temperature. The resulted precipitate was filtered, and the collected crystals were washed with 10 ml ethyl acetate followed by to give 4.15 g Cinacalcet fumarate. [See X-ray powder diffraction in FIG. 1] The ¹H and ¹³C NMR spectra are depicted in FIGS. 3, and 4.

### Preparation of a Crystalline Form of Cinacalcet Succinate.

### Example 2

3.97 g cinacalcet hydrochloride (10 mmole) was stirred with 20 ml of NaHCO₃ (5% in water) and 10 ml of ethyl acetate at room temperature for 1 hour. The layers were separated and the aqueous solution was further extracted twice with 5 ml ethyl acetate each. The combined organic solution was passed through a Hi-flow pad, then 1.14 g succinic acid was then added, and the mixture was heated over a hot plate (45° C. to 55° C.) for few minutes until all solids dissolved. The reaction mixture was stirred at room temperature for 1 hour, and then the solvent was evaporated using rotavap until the material became a thick paste. Upon adding 15 ml diethylether and scratching the vessel walls, the thick paste became free flowing suspension.

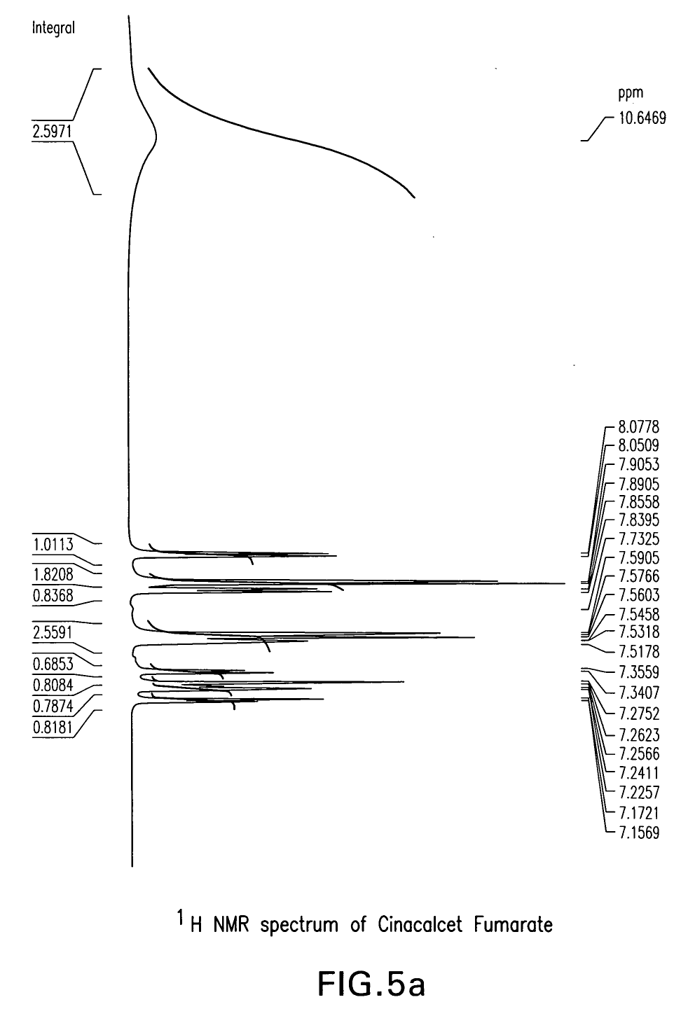

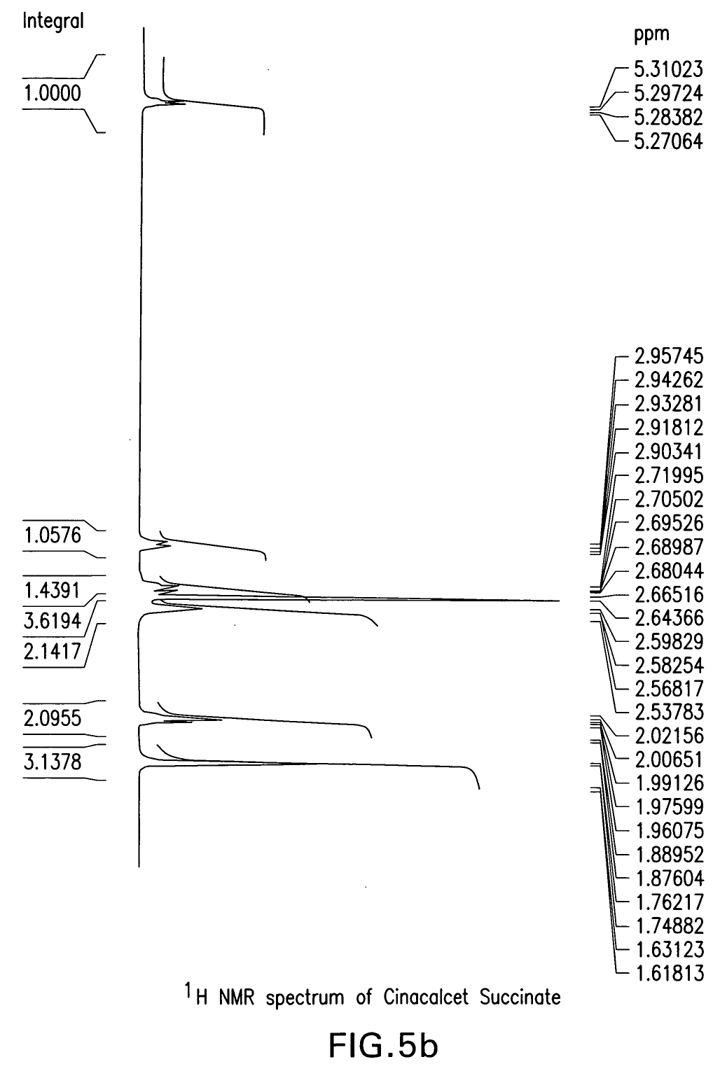

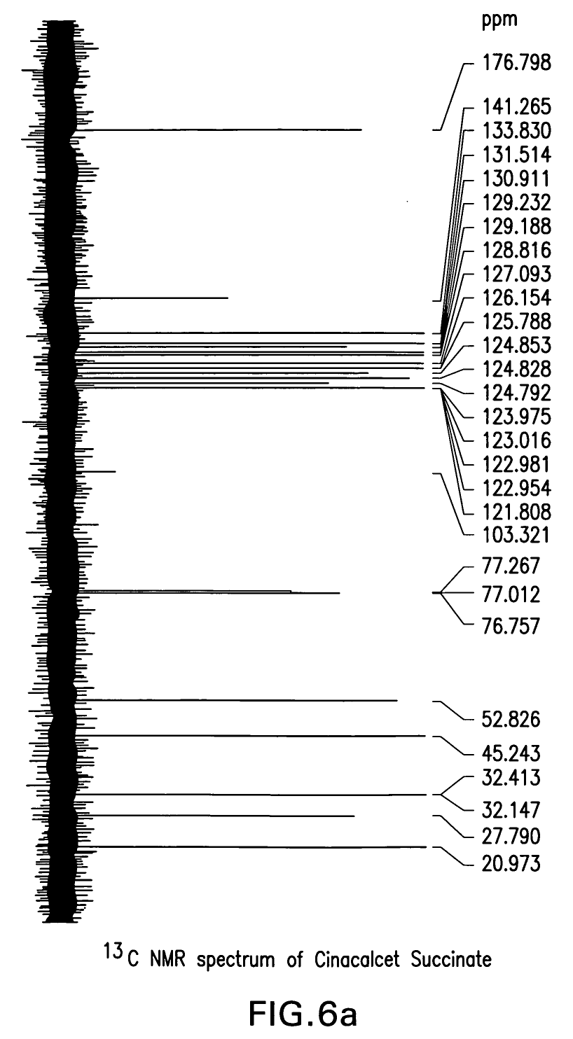

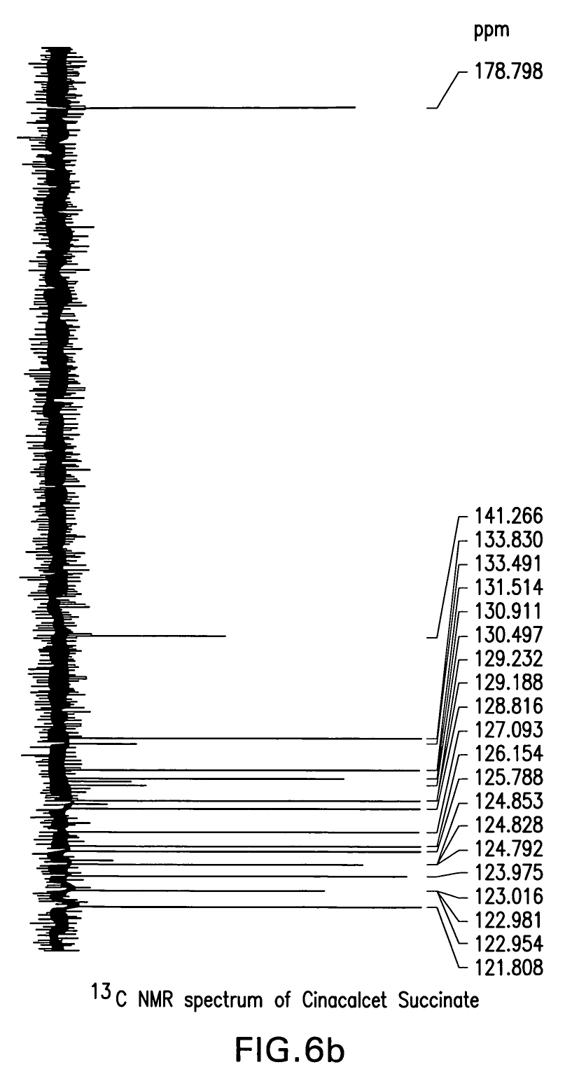

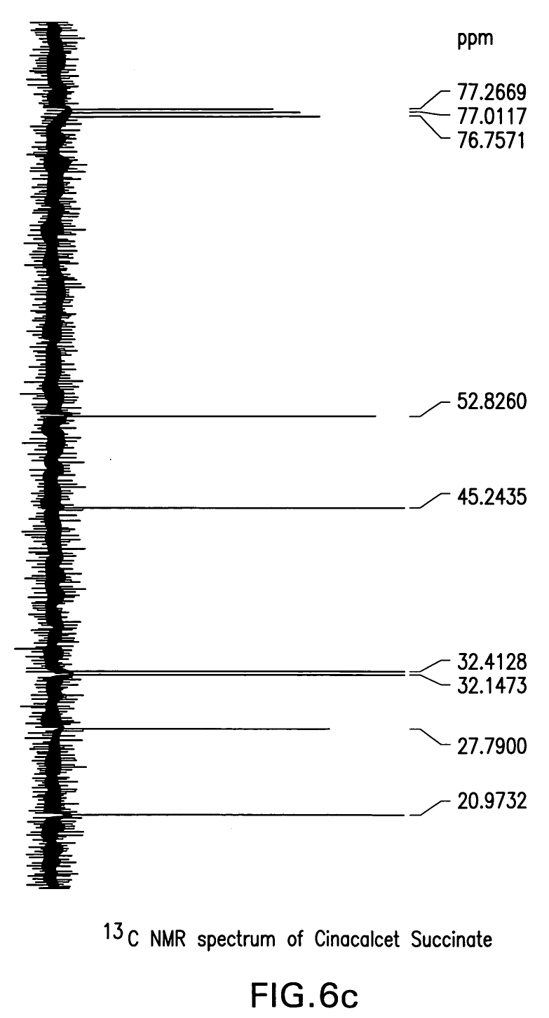

Thereafter, the solids were collected using coarse filter and the solid powder was dried under vacuum to give 3.34 g. [See X-ray powder diffraction in FIG. 2] The ¹H and ¹³C NMR spectra are depicted in FIGS. 5, and 6.

What is claimed:

1. Cinacalcet Fumarate.

2. The Cinacalcet Fumarate of claim 1, wherein it is solid Cinacalcet Fumarate.

3. The Cinacalcet Fumarate of claim 2, wherein it is crystalline Cinacalcet Fumarate.

4. The Cinacalcet Fumarate of claim 3, characterized by data selected from at least one of a powder XRD pattern with two peaks at about 7.5 and 19.4±0.2 degrees two-theta and three peaks selected from a list of five peaks at about 11.6, 12.6, 16.3, 17.2 and 25.4±0.2 degrees two-theta; and a powder XRD pattern having main peaks at about 7.5, 15.1, 16.3, 18.1° 2θ±0.2° 2θ.

5. The crystalline form of Cinacalcet Fumarate of claim 4, further characterized by a powder XRD peak at about 24.5° 2θ±0.2° 2θ.

6. The crystalline form of Cinacalcet Fumarate of claim 3, further characterized by a PXRD pattern as illustrated in FIG. 1.

7. A process for preparing the crystalline form of Cinacalcet Fumarate comprising reacting cinacalcet with fumaric acid.

8. The process of claim 7, wherein the reaction is carried out in an organic solvent selected from the group consisting of C₃ to C₇ esters, C₁ to C₆ chlorinated hydrocarbons, and C₆ to C₁₂ aromatic hydrocarbons.

9. The process of claim 8, wherein the organic solvent is selected from the group consisting of ethyl acetate, dichloromethane, toluene, and chloroform.

10. The process of claim 9, wherein the solvent is ethyl acetate.

11. The process of claim 7, wherein the process comprises a preliminary step of neutralizing a Cinacalcet salt with a base.

12. The process of claim 11, wherein the base is an inorganic base.

13. The process of claim 12, wherein the base is selected from the group consisting of an alkali metal hydrogen carbonate, an alkali metal carbonate, and an alkali hydroxide.

14. The process of claim 13, wherein the base is sodium bicarbonate.

15. The process of claim 11, wherein the Cinacalcet salt is Cinacalcet HCl, Cinacalcet HBr, Cinacalcet sulfate, or Cinacalcet phosphate.

16. The process of claim 15, wherein the Cinacalcet salt is Cinacalcet HCl.

17. The process of claim 7, further comprising recovering crystals of Cinacalcet Fumarate.

18. The process of claim 7, wherein the process is carried out in one pot.

19. The process of claim 7, wherein the molar ratio of fumaric acid to cinacalcet is preferably about 1:1 to about 1:1.2.

20. The process of claim 7, wherein the ratio of organic solvent to cinacalcet base is preferably about 5:1 to about 7:1 (v/w).

21. The process of claim 7, wherein the process comprises:
a) reacting a cinacalcet salt with a base in an aqueous reaction mixture to obtain cinacalcet base;
b) combining the reaction mixture with a water immiscible organic solvent to obtain two phases, wherein the combining can be carried out before or after formation of the cinacalcet base;
c) combining the two phase reaction mixture with fumaric acid to obtain cinacalcet fumarate, with the proviso that the fumaric acid can optionally be dissolved in the solvent of step b) and added in step b);
d) recovering the cinacalcet fumarate.

22. Cinacalcet succinate.

23. The Cinacalcet succinate of claim 22, wherein the Cinacalcet succinate is solid.

24. The Cinacalcet succinate of claim 22, wherein the Cinacalcet succinate is crystalline.

25. The crystalline form of Cinacalcet Succinate, characterized by data selected from at least one of a powder XRD pattern with two peaks at about 6.7 and 18.9±0.2 degrees two-theta and three peaks selected from a list of five peaks at about 7.2, 10.7, 14.4, 16.3 and 17.0±0.2 degrees two-theta; and a powder XRD pattern with main peaks at about 6.7, 7.2, 13.5, 15.1° 2θ±0.2° 2θ.

26. The crystalline form of Cinacalcet Succinate according to claim 25, further characterized by a PXRD peak at about 18.5° 2θ±0.2° 2θ.

27. The crystalline form of Cinacalcet Succinate of claim 24, further characterized by a PXRD pattern as illustrated in FIG. 2.

28. A process for preparing Cinacalcet Succinate comprising reacting cinacalcet with succinic acid, while heating the reaction mixture.

29. The process of claim 28, wherein the process first comprises neutralizing a Cinacalcet salt with a base.

30. The process of claim 29, wherein the base is an inorganic base.

31. The process of claim 30, wherein base is selected from the group consisting of an alkali metal hydrogen carbonate, an alkali metal carbonate, and an alkali hydroxide.

32. The process of claim 31, wherein base is sodium carbonate.

33. The process of claim 32, wherein base is aqueous NaHCO₃.

34. The process of claim 29, wherein the Cinacalcet salt is Cinacalcet HCl, Cinacalcet HBr, Cinacalcet sulfate, or Cinacalcet phosphate.

35. The process of claim 34, wherein the Cinacalcet salt is Cinacalcet HCl.

36. The process of claim 28, wherein the reaction is carried out in a water immiscible organic solvent.

37. The process of claim 36, wherein the organic solvent is selected from the group consisting of C₃ to C₇ esters, C₁ to C₆ chlorinated hydrocarbons, and C₆ to C₁₂ aromatic hydrocarbons.

38. The process of claim 37, wherein the solvent is selected from the group consisting of ethyl acetate, dichloromethane, toluene, and chloroform.

39. The process of claim 38, wherein the solvent is ethyl acetate.

40. The process of claim 28, wherein the cinacalcet succinate is recovered from the reaction mixture.

41. The process of claim 28, wherein the process is carried out in one pot.

42. The process of claim 28, wherein the ratio of succinic acid to cinacalcet is preferably about 1:1 to about 1:1.2.

43. The process of claim 28, wherein the ratio of organic solvent to cinacalcet base is preferably about 5:1 to about 7:1 (v/w).

44. The process of claim 28, wherein the process comprises:
a) reacting a cinacalcet salt with a base in an aqueous reaction mixture to obtain cinacalcet base;
b) combining the reaction mixture with a water immiscible organic solvent to obtain two phases, wherein the combining can be carried out before or after formation of the cinacalcet base;
c) combining the two phase reaction mixture with succinic acid to obtain cinacalcet succinate, with the proviso that the succinic acid can optionally be dissolved in the solvent of step b) and added in step b);
d) recovering the cinacalcet succinate.

45. A pharmaceutical composition, comprising a crystalline form of Cinacalcet Fumarate and/or Cinacalcet Succinate and at least one pharmaceutically acceptable excipient.

46. A process for preparing the pharmaceutical composition on claim 45, comprising combining a crystalline form of Cinacalcet Fumarate and/or a crystalline form of Cinacalcet Succinate with at least one pharmaceutically acceptable excipient.

47. A method of treating secondary hyperparathyroidism comprising administering comprising administering the pharmaceutical composition of claim 45 to a human.

48. The method of claim 48, wherein the secondary hyperparathyroidism is associated with chronic kidney disease.

49. The process of claim 7, wherein the crystalline form is cinacalcet Fumarate.

50. The process of claim 28, wherein the crystalline form is Cinacalcet succinate
